(19) United States
(12) Patent Application Publication
(10) Pub. No.: US 2023/0050835 A1
(43) Pub. Date: Feb. 16, 2023

(54) COSMETIC COMPOSITION

(71) Applicant: L'ORÉAL, Paris (FR)

(72) Inventors: Hubert Tunchiao LAM, Berkeley Heights, NJ (US); Lynne A. MCCULLOUGH, Brooklyn, NY (US); Yan L. LEE, Elmwood Park, NJ (US); Jialu GU, Cliffside Park, NJ (US)

(73) Assignee: L'ORÉAL, Paris (FR)

(21) Appl. No.: 17/387,420

(22) Filed: Jul. 28, 2021

(51) Int. Cl. A61K 8/892; A61K 8/891; A61K 8/85; A61K 8/02; A61Q 1/06

(52) U.S. Cl. CPC A61K 8/892; A61K 8/891; A61K 8/85; A61K 8/0229; A61Q 1/06; A61K 2800/412; A61K 2800/31; A61K 2800/30; A61K 2800/43

(57) ABSTRACT

Anhydrous cosmetic compositions that provide improved dispersion of pigments and fillers, and solids spreading, as well as providing a matte color or mattifying effect. The anhydrous cosmetic compositions comprise a polyhydroxyalkanoate; a polysiloxane elastomer; and a filler, a pigment, or both.

### FIELD OF THE INVENTION

The present invention relates to cosmetic compositions, and specifically to cosmetic lip compositions utilizing polyhydroxyalkanoates and silicone elastomers to provide improved pigment and filler dispersion and spreading.

### BACKGROUND

Many cosmetic products, and particularly lipsticks, are designed to allow a user to apply a colorant or pigment to lips, skin, etc. while maintaining a good level of skin feel and comfort of wearing the cosmetic product. Often, cosmetic chemists must balance tradeoffs in properties of such products, such as between dispersibility and viscosity or moldability as well as good mechanical properties. A solid lipstick may be moldable, into which the powders and colorants are not easily dispersible, while a liquid lip product containing the same may be far more dispersible, but not moldable. Such tradeoffs lead to inferior products.

Thus, the ability to improve the dispersion pigments and fillers, and to improve solid spreading, in cosmetics, even in solid, waxy, or pasty cosmetic products, is therefore useful and desirable while maintaining a good level of glide, skin feel and comfort.

### BRIEF SUMMARY

Disclosed is an anhydrous cosmetic composition that provides improved pigment and filler powders dispersion and spreading, including when formulated as a solid, waxy, or pasty cosmetic product (e.g., a traditional lipstick).

The disclosed anhydrous composition generally requires three components: (i) a polyhydroxyalkanoate; (ii) a polysiloxane elastomer; and (iii) a filler, a pigment, or both. Advantageously, the disclosed composition is in the form of a solid lipstick.

The polyhydroxyalkanoate(s) may be present in the composition in an amount of, e.g., between 1% and 5% by weight of the composition. Advantageously, the polyhydroxyalkanoate comprises (i) a polyhydroxybutyrate, (ii) polyhydroxy pentanoate (ii) poly [(R)-3-hydroxyhexanoate](PHHx), (iv) a crosspolymer comprising monomers from hydroxybutyric acid, hydroxypentanoate acid, and/or hydroxyhexanoate, or (v) a combination thereof or derivatives thereof.

The polysiloxane elastomer may be present in an amount of, e.g., between 1% and 5% by weight of the composition. Advantageously, the polysiloxane elastomer consists of an elastomeric crosslinked organopolysiloxane powder coated with silicone resin (such as a silsesquioxane resin).

The cosmetic composition may optionally be free or substantially free of some materials, or classes of materials. For example, advantageously, the cosmetic composition may be free or substantially free of UV filters, polyamides, acrylic polymers, hydrophilic synthetic amorphous silica particles, and/or cellulose or ethylcellulose-based polymer particles.

Advantageously, the cosmetic composition also includes a silicone crosspolymer in amount of between, e.g., 5% and 30% by weight of the composition.

Advantageously, the cosmetic composition also includes a wax and a hydrocarbon oil.

Advantageously, the cosmetic composition may consist, or consist essentially of: the polyhydroxyalkanoate, the polysiloxane elastomer, the pigment; and optionally a filler, an antioxidant, a fragrance, a colorant, a wax, a hydrocarbon, an additional polymer, a fatty ester, a non-volatile silicone, or a combination thereof.

Also disclosed is a method for providing matte color or effect to lips, by first providing an anhydrous cosmetic composition comprising a polyhydroxyalkanoate; a polysiloxane elastomer; and a pigment, filler, or both, and then applying the anyhydrous cosmetic composition to lips.

### DETAILED DESCRIPTION

As used herein, articles such as “a” and “an” when used in a claim, are understood to mean one or more of what is claimed or described.

As used herein, the expression “at least one” means one or more and thus includes individual components as well as mixtures/combinations.

All percentages listed are by weight unless otherwise noted.

As used herein, the term “about [a number]” is intended to include values rounded to the appropriate significant digit. Thus, “about 1” would be intended to include values between 0.5 and 1.5, whereas “about 1.0” would be intended to include values between 0.95 and 1.05.

As used herein, the terms “include”, “includes” and “including” are meant to be non-limiting.

As used herein, the term “substantially free [of an ingredient]” means that the composition contains less than 1% of the identified ingredient. The term “free [of an ingredient]” means the composition contains less than 0.1% of the identified ingredient.

As used herein, “substituted” means comprising at least one substituent. Non-limiting examples of substituents for substitution include atoms, such as oxygen atoms and nitrogen atoms, as well as functional groups, such as hydroxyl groups, ether groups, alkoxy groups, acyloxyalkyl groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

For purposes of the compositions and methods of the present invention where the invention “consists essentially of” the identified ingredients and/or process steps, the “basic and novel properties” of such compositions and/or methods are “improved dispersion of pigments and/or fillers”, “improved solid spreading”, and “providing a matte or mattifying finish.”

Disclosed is an anhydrous composition that generally requires at least three components: (i) a polyhydroxyalkanoate (PHA); (ii) a polysiloxane elastomer; and (iii) a filler, a pigment, or both. The composition may comprise any form, although preferably the composition is in the form of a solid, waxy, or pasty cosmetic product, such as a solid, moldable lipstick.

PHA

The cosmetic composition generally includes at least one polyhydroxyalkanoate (PHA). PHAs are known to be biodegradable and can be produced via, e.g., environmentally friendly fermentation processes.

Surprisingly, PHAs can substitute for more traditional polymer powders (such as PMMA or Nylon) in various bases to provide equivalent viscosity modification, mattifying effects, and sebum transfer resistance.

The polyhydroxyalkanoates drive the improvements in volume and removability and are biodegradable. As is known in the art, PHAs are a sub-family of polyesters, which can be made with hydroxyl carboxylic acid monomers where the various monomers have one or more carbon chain lengths. Surprisingly, not all polyhydroxyalkanoates can perform adequately in the disclosed compositions.

Here, the monomers of the polyhydroxyalkanoate should be in the form of a powder. This will preferably involve PHAs comprised of at least one monomer that has a carbon chain length of between 3 and 7 and more specifically between 4 and 6. Preferred PHAs utilize short chain length monomers, and are based on carboxylic acids having 3 carbons (propionic or propanoic acid), 4 carbons (butyric or butanoic acid), 5 carbons (valeric or pentanoic acid), 6 carbons (caproic or hexanoic acid), or 7 carbons (enanthic or heptanoic acid).

Non-limiting examples of monomers that can form the PHAs may generally have the formula:

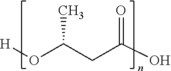

where n=1 to 6. In some embodiments, only a single monomer is present. In other embodiments, different monomers are utilized; in some embodiments, one monomer has n=4 and at least one other monomer has n=1, 2, 3, 4, 5 or 6.

Polyhydroxybutyrate (PHB or PH3B). Polyhydroxybutyrate is a polymer of 3 hydroxybutyric acid, and conforms to the formula represented by the structure shown below:

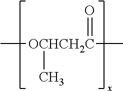

This is also known as: Butanoic acid, 3-hydroxy-, (3R)-, homopolymer, and is, available commercially as, e.g., PHB from Metabolix or TianAn Biopolymer or Eckart or MicroPowders

Hydroxybutyric Acid/Hydroxypentanoic Acid Copolymer (PHB-PHV copolymer). Hydroxybutyric Acid/Hydroxypentanoic Acid Copolymer is the copolymer that conforms generally to the structure shown below.

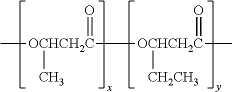

This is also known as: Pentanoic acid, 3-hydroxy-, (3R)-, polymer with (3R)-3-hydroxybutanoic acid, and has an INCI name of Hydroxybutyric Acid/Hydroxypentanoic copolymer.

Poly(3-hydroxybutyrate-co-3-hydroxyvalerate) (PHBV). Poly(3-hydroxybutyrate-co-3-hydroxyvalerate) is the copolymer of hydroxybutyric acid and hydroxypentanoic acid that conforms generally to the structure below:

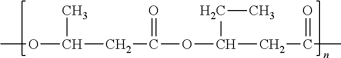

Various suitable PHAs can be obtained from Eckart (a member of the Altana group), Tianan Enmat or MicroPowders.

Other non-limiting examples of suitable polyhydroxyalkanoates include poly-3-hydroxypropionate (P3HP), poly-3-hydroxyvalerate (PHV), and poly-4-hydroxybutyrate (P4HB).

In some embodiments, the chain length of the monomeric constituents of the PHA may have a chain length between 1 and 6 carbons. In preferred embodiments, the PHA has a chain length between 4 and 6 carbons, such as between 3 and 5 carbons, such as between 4 and 5 carbons, or such as 4 carbons. In some embodiments, the PHA has a chain length between 4 and 6 carbons. All ranges are inclusive of endpoints.

In certain embodiments, the PHA is poly-3-hydroxybutyrate (PHB), poly-3-hydroxyvalerate (PHV), poly [(R)-3-hydroxypentanoate] (PHHP), poly [(R)-3-hydroxyhexanoate] (PHHx), poly (3-hydroxybutyrate-co-3-hydroxyvalerate) (PHBV), poly (3-hydroxybutyrate-co-3-hydroxyexanoate) (PHBH), poly (3-hydroxybutyrate-co-4-hydroxybutyrate), poly(3-hydroxybutyrate-co-3-hydroxyvalerate-co-4-hydroxyvalerate) (PHBW), or a combination thereof.

The PHA may be a crosspolymer comprising monomers from hydroxybutyric acid, hydroxypentanoic acid, and/or hydroxyhexanoic acid.

The PHA may be a part of a copolymer, or the copolymer may be derived from two or more PHAs.

In some embodiments, the composition comprises only a single PHA. In other embodiments, the composition comprises a plurality of PHAs.

Preferably, the PHA is PHB, such as PHB available from Micropowders sold under the trade name BIOSOFT 915. The PHA can also be a Hydroxybutyric Acid/Hydroxypentanoic Acid Copolymer sold under the trade name Ecofil B110 from. Eckart America or 3-Hydroxybutyrate/3-Hydroxyhexanoate copolymer sold under the trade name Techpolymer EFA-20X from Sekisui Plastics Co. and PHB particles from Tianan from the ENMAT™ brand.

In certain embodiments, the total amount of PHA present in the composition is ≥0.01%, ≥0.1%, ≥0.5%, ≥1%, or ≥2% by weight of the composition.

In certain embodiments, the total amount of PHA present in the composition is ≤50%, ≤30%, ≤15%, ≤8%, ≤5%, or ≤3% by weight of the composition

In preferred embodiments, the total amount of PHA present (x_{PHA}) in the composition is 0.1%≤x_{PHA≤}30% by weight of the composition. In more preferred embodiments, 1%≤x_{PHA}≤8%. In still more preferred embodiments, 1%≤x_{PHA}≤5%.

In the context of the present invention and of the appended claims, by “average particle diameter” it is meant particularly according, unless otherwise indicated, the diameter d50 (median value), that is, the value of the diameter below which 50% by weight of the particle population is found (see “A Guidebook to Particle Size Analysis “published by Horiba Instruments Inc. 2016, available at www.horiba.corn/fileadmin/uploads/Scientific/eMag/PSA/Guidebook/pdf/PSA_Guide book.pdf). It can be determined by laser diffraction technique, according to the ISO 13320:2008 standard or determined with TEM Transmission Electron Microscopy.

In certain embodiments, the PHA is in the form of particles having an average diameter (d50) less than 100 μm. In certain embodiments, the d50 is less than 50 μm. In certain embodiments, the d50 is less than 30 μm. In certain embodiments, the d50 is less than 20 μm. In certain embodiments, the d50 is greater than 0.1 nm. In certain embodiments, the d50 is greater than 1 nm. In certain embodiments, the d50 is greater than 100 nm. In certain embodiments, the d50 is greater than 1 μm. In preferred embodiments, the d50 is between 0.1 nm and 50 μm, and more preferably between 1 μm and 20 μm. In some embodiments, the d50 is between 8 μm and 12 μm. In a preferred embodiment, the d50 is 10 μm.

In certain embodiments, the PHA is selected to have an oil absorption of between 60 to 150 grams of one or more desired oils per 100 grams of PHA. In certain embodiments, the desired one or more oils is natural or synthetic sebum. In certain embodiments, the desired one or more oils is a cosmetic oil present in the composition, such as a silicone, an ester, a fragrance, or an antioxidant.

Polysiloxane Elastomer

The cosmetic composition also contains a polysiloxane elastomer, preferably in the form of a polysiloxane particle.

In preferred embodiments, the polysiloxane elastomer is a spherical elastomer, and preferably a non-emulsifying elastomer. In more preferred embodiments, the spherical non-emulsifying elastomer silicone is provided as an elastomeric crosslinked organopolysiloxane powder coated with silicone resin, and particularly a silsesquioxane resin coating. An example of such a spherical non-emulsifying elastomer silicone is Vinyl Dimethicone/Methicone Silseqsquioxane Crosspolymer, available commercially from Shin-Etsu, including, e.g., KSP-100 (5 μm average diameter particles), KSP-101 (12 μm average diameter particles), KSP-102 (30 μm average diameter particles), and KSP-105 (2 μm average diameter particles).

In some embodiments, additional polysiloxane elastomers are utilized.

In some embodiments, the additional polysiloxane elastomer comprises, e.g., polydimethylsiloxane, particularly crosslinked polydimethylsiloxane.

In some embodiments, the additional polysiloxane elastomer comprises, e.g., non-spherical elastomer particles in a gel form, such as a silicone elastomer may be chosen from Dimethicone Crosspolymer (INCI name), Vinyl Dimethicone Crosspolymer (INCI name), Dimethicone/Vinyl Dimethicone Crosspolymer (INCI name) or Dimethicone Crosspolymer-3 (INCI name), dispersed in a linear silicone or cyclic oil. Mention may be made especially of the compounds having the following INCI names: dimethicone/vinyl dimethicone crosspolymer, such as USG-105® and USG-107A® from the company Shin-Etsu; DC9506® and DC97018 from the company Dow Corning; dimethicone/vinyl dimethicone crosspolymer (and) dimethicone, such as KSG-6® and KSG-16® from the company Shin-Etsu; dimethicone/vinyl dimethicone crosspolymer (and) cyclopentasiloxane, such as KSG-15®; cyclopentasiloxane (and) dimethicone crosspolymer, such as DC9040®, DC9045® and DC5930® from the company Dow Corning; dimethicone (and) dimethicone crosspolymer, such as DC9041® from the company Dow Corning; dimethicone (and) dimethicone crosspolymer, such as Dow Corning EL-9240® Silicone Elastomer Blend from the company Dow Corning (mixture of polydimethylsiloxane crosslinked with hexadiene/polydimethylsiloxane (2 cSt)); and C4-24 alkyl dimethicone/divinyl dimethicone crosspolymer, such as NuLastic Silk MA® from the company Alzo.

In certain embodiments, the total amount of polysiloxane elastomer present in the composition is ≥0.01%, ≥0.1%, ≥0.5%, or ≥1% by weight of the composition.

In certain embodiments, the total amount of polysiloxane elastomer present in the composition is ≤8%, ≤5%, ≤4%, ≤3%, or ≤2% by weight of the composition.

In preferred embodiments, the total amount of polysiloxane elastomer present (x_{PSE}) in the composition is 0.1%≤x_{PSE}≤8% by weight of the composition. In more preferred embodiments, 1%≤x_{PSE}≤5%.

In some embodiments, the total amount of spherical non-emulsifying polysiloxane elastomer present (x_{SNEPSE}) in the composition is 0.1%≤x_{SNEPSE}≤8% by weight of the composition. In more preferred embodiments, 1%≤x_{SNEPSE}≤5%.

In some embodiments, the total amount of spherical non-emulsifying polysiloxane elastomer present (x_{SNEPSE}) in the composition is 1%≤x_{SNEPSE}≤5% and the total amount of additional polysiloxane elastomer is between 1% and 30%, 10% and 30%, or 20% and 30% by weight of the composition.

The cosmetic composition also contains a pigment, a filler, or both.

Pigment

The cosmetic compositions may contain at least one cosmetically acceptable pigment. Examples of suitable pigments include, but are not limited to, inorganic pigments, organic pigments, lakes, pearlescent pigments, iridescent or optically variable pigments, and mixtures thereof. A pigment should be understood to mean inorganic or organic, white or colored particles. Said pigments may optionally be surface-treated within the scope of the present invention but are not limited to treatments such as silicones, perfluorinated compounds, lecithin, and amino acids.

Representative examples of inorganic pigments useful in the present invention include those selected from the group consisting of rutile or anatase titanium dioxide, coded in the Color Index under the reference CI 77,891; black, yellow, red and brown iron oxides, coded under references CI 77,499, 77, 492 and, 77,491; manganese violet (CI 77,742); ultramarine blue (CI 77,007); chromium oxide (CI 77,288); chromium hydrate (CI 77,289); and ferric blue (CI 77,510) and mixtures thereof.

Representative examples of organic pigments and lakes useful in the present invention include, but are not limited to, D&C Red No. 19 (CI 45,170), D&C Red No. 9 (CI 15,585), D&C Red No. 21 (CI 45,380), D&C Orange No. 4 (CI 15,510), D&C Orange No. (CI 45,370), D&C Red No. 27 (CI 45,410), D&C Red No. 13 (CI 15,630), D&C Red No. 7 (CI 15,850), D&C Red No. 6 (CI 15,850), D&C Yellow No. 5 (CI 19,140), D&C Red No. 36 (CI 12,085), D&C Orange No. 10 (CI 45,425), D&C Yellow No. 6 (CI 15,985), D&C Red No. 30 (CI 73,360), D&C Red No. 3 (CI 45,430) and the dye or lakes based on cochineal carmine (CI 75,570) and mixtures thereof.

Representative examples of pearlescent pigments useful in the present invention include those selected from the group consisting of the white pearlescent pigments such as mica coated with titanium oxide, mica coated with titanium dioxide, bismuth oxychloride, titanium oxychloride, colored pearlescent pigments such as titanium mica with iron oxides, titanium mica with ferric blue, chromium oxide and the like, titanium mica with an organic pigment of the above-mentioned type as well as those based on bismuth oxychloride and mixtures thereof.

The precise amount and type of pigment employed in embodiments of the disclosed compositions will depend on the color, intensity and use of the cosmetic composition and, as a result, will be determined by those skilled in the art of cosmetic formulation.

In certain embodiments, the total amount of pigment present in the composition is ≥0.1%, ≥1%, ≥3%, ≥5%, ≥8%, or ≥10% by weight of the composition.

In certain embodiments, the total amount of pigment present in the composition is ≤40%, ≤30%, ≤25%, ≤20%, or ≤15% by weight of the composition.

In preferred embodiments, the total amount of pigment present (x_{PIGMENT}) in the composition is 1%≤x_{PIGMENT}≤30% by weight of the composition. In more preferred embodiments, 5%≤x_{PIGMENT}≤25%.

Filler

The cosmetic composition according to the present invention may further comprise at least one filler.

As used herein, the term “filler” should be understood as meaning colorless natural or synthetic particles of any shape which are insoluble in the medium of the composition, whatever the temperature at which the composition is manufactured. Thus, the filler is different from the pigment described above.

The fillers may be inorganic or organic and of any shape (for instance, platelet, spherical, and oblong shapes) and with any crystallographic form (for example, sheet, cubic, hexagonal, orthorhombic, and the like). Examples of suitable additional fillers include, but are not limited to, talc; mica; silica; kaolin; powders of polyamide such as Nylon®; poly-β-3-alanine powders; polyethylene powders; polyurethane powders, such as the powder formed of hexamethylene diisocyanate and trimethylol hexyllactone copolymer sold under the name Plastic Powder D-400 by Toshiki; the powders formed of tetrafluoroethylene polymers (Teflon®); lauroyllysine; starch; boron nitride; polymeric hollow microspheres, such as microspheres of poly(vinylidene chloride)/acrylonitrile, for example Expancel® (Nobel Industries), and microspheres of acrylic acid copolymers; silicone resin powders, for example, silsesquioxane powders (for instance, silicone resin powders disclosed in European Patent No. 0 293 795 and Tospearls® from Toshiba); poly(methyl methacrylate) particles; precipitated calcium carbonate; magnesium carbonate; basic magnesium carbonate; hydroxyapatite; hollow silica microspheres; glass microcapsules; ceramic microcapsules; metal soaps derived from organic carboxylic acids comprising from 8 to 22 carbon atoms, for example, from 12 to 18 carbon atoms, such as zinc stearate, magnesium stearate, lithium stearate, zinc laurate, and magnesium myristate; barium sulphate; and mixtures thereof.

In certain embodiments, the total amount of filler present in the composition is ≥0.1%, ≥1%, ≥3%, ≥5%, ≥8%, or ≥10% by weight of the composition.

In certain embodiments, the total amount of filler present in the composition is ≤80%, ≤70%, ≤60%, ≤50%, ≤40%, ≤30%, ≤25%, ≤20%, ≤15%, ≤10%, or ≤5% by weight of the composition.

In some embodiments, the total amount of filler present (x_{FILLER}) in the composition is 1%≤x_{FILLER}≤25% by weight of the composition. In preferred embodiments, 1%≤x_{FILLER}≤15%. In more preferred embodiments, 1%≤x_{FILLER}≤5%. In another preferred embodiments, 5%≤x_{FILLER}≤10%.

In some embodiments, the anhydrous cosmetic composition according to claim 1, wherein the anhydrous cosmetic composition may consist or consist essentially of: (1) the polyhydroxyalkanoate; (2) the polysiloxane elastomer; (3) the pigment; and (4) optionally (a) the filler, (b) an antioxidant, (c) a fragrance, (d) a non-pigment colorant (such as an oil-soluble dye), (e) a wax, (f) a hydrocarbon, (g) an additional polymer, (h) a fatty ester, (i) a non-volatile silicone, or a combination thereof. For example, in some embodiments, the cosmetic composition further comprises a silicone crosspolymer. In other embodiments, the cosmetic composition further comprises a wax and a hydrocarbon oil. In still other embodiments, the cosmetic composition comprises a silicone crosspolymer, a wax, and a hydrocarbon oil.

In some embodiments, the anhydrous cosmetic composition consists or consists essentially of the polyhydroxyalkanoate, the polysiloxane elastomer, the pigment, and optionally the filler, an antioxidant, a fragrance, a non-pigment colorant, a wax, a hydrocarbon, an additional polymer, a fatty ester, a non-volatile silicone, or a combination thereof. In another example, no additional polymer is present, and the composition consists or consists essentially of the polyhydroxyalkanoate, the polysiloxane elastomer, the pigment, the filler, an antioxidant, a fragrance, a non-pigment colorant, a wax, a hydrocarbon, a fatty ester, and a non-volatile silicone. In other embodiments, no additional polymer or non-pigment colorant is present, and the composition consists or consists essentially of the polyhydroxyalkanoate, the polysiloxane elastomer, the pigment, the filler, an antioxidant, a fragrance, a wax, a hydrocarbon, a fatty ester, and a non-volatile silicone.

Wax

“Wax” as used herein means a lipophilic compound that is solid at room temperature (25° C.), with a solid/liquid reversible change of state, having a melting point of greater than or equal to 30° C., which may be up to 200° C., and in particular up to 120° C., including all ranges and subranges therebetween. Preferred waxes are those having a melting point of greater than or equal to 45° C. and in particular greater than or equal to 55° C.

Preferred waxes are chosen from waxes that are solid at room temperature of animal, plant, mineral and/or synthetic origin, and mixtures thereof. Preferably, the waxes are hydrocarbon-based or silicone waxes “Hydrocarbon-based wax” means a wax formed essentially from, or even constituted by, carbon and hydrogen atoms, and possibly oxygen and nitrogen atoms, and not containing any silicon or fluorine atoms. It may contain groups such as, for example, alcohol, ester, ether, carboxylic acid, amine and/or amide groups. “Silicone wax” means compounds comprising at least one silicon atom, preferably comprising Si—O groups.

In some embodiments, the wax comprises a polar wax.

In some embodiments, the polar wax is hydrocarbon-based. Suitable hydrocarbon-based waxes include, for example, beeswax, lanolin wax, rice bran wax, carnauba wax, candelilla wax, shellac wax; montan wax, orange wax and lemon wax, laurel wax and olive wax.

In some embodiments, the polar wax is an ester wax. “Ester wax” means a wax comprising at least one ester function or group. Examples of ester waxes include:

i) the waxes of formula R1COOR2 in which R1 and R2 represent linear, branched or cyclic aliphatic chains in which the number of atoms ranges from 10 to 50, which may contain a heteroatom such as 0, N or P, and whose melting point ranges from about 25 to about 120° C., such as for example C20-C40 alkyl (hydroxystearyloxy)-stearate (the alkyl group containing from 20 to 40 carbon atoms), alone or as a mixture, or a C20-C40-alkyl stearate, Specific examples of such waxes are sold under the names Kester Wax K 82 P®, Hydroxypolyester K 82 P®, Kester Wax K 80 P®. or Kester Wax K82H by the company Koster Keunen. Other specific examples include esters of polyethylene glycol and of montanic acid (octacosanoic acid), such as the wax Licowax KPS Flakes (INCI name: glycol montanate) sold by the company Clariant.

ii) bis(1,1,1-trimethylolpropane) tetrastearate, sold under the name Hest 2T-4S® by the company Heterene,

iii) waxes of diesters of a dicarboxylic acid of general formula R3—(—OCO—R4—COO—R.5), in which R3 and R5 are identical or different, preferably identical, and represent a C4-C30 alkyl group (alkyl group comprising from 4 to 30 carbon atoms) and R4 represents a linear or branched C4-C.30 aliphatic group (alkyl group comprising from 4 to 30 carbon atoms), which may or may not contain one or more unsaturations, and is preferably linear and unsaturated,

iv) waxes obtained by hydrogenation of castor oil esterified with cetyl alcohol, sold under the names Phytowax ricin 16L64® and 22L73® by the company Sophim, and

v) hydrogenated oils such as hydrogenated jojoba oil.

In some embodiments, the polar wax is an oil-soluble polar modified polymer. “Polar modified polymer” as used herein refers to a hydrophobic homopolymer or copolymer which has been modified with hydrophilic unit(s). “Oil-soluble” as used herein means that the polar modified polymer is soluble in oil, and may be, e.g., a high carbon oil-soluble polar modified polymer, or a low carbon oil-soluble polar modified polymer.

“High carbon oil-soluble polar modified polymer” means a polymer containing more than 20 carbon atoms. Suitable monomers for the hydrophobic homopolymers and/or copolymers include, but are not limited to, cyclic, linear or branched, substituted or unsubstituted, C22-C40 compounds such as, C22-C28 compounds, C24-C26 compounds, C26-C28 compounds, and C30-C38 compounds, including all ranges and subranges therebetween. Preferably, the monomers are C24-26 compounds, C26-C28 compounds or C30-C38 compounds.

“Low carbon oil-soluble polar modified polymer” means a polymer containing 2 to 20 carbon atoms. Suitable monomers for the hydrophobic homopolymers and/or copolymers include, but are not limited to, cyclic, linear or branched, substituted or unsubstituted, C2-C20 compounds such as, for example, styrene, ethylene, propylene, isopropylene, butylene, isobutylene, pentene, isopentene, isoprene, hexene, isohexene, decene, isodecene, and octadecene, including all ranges and subranges therebetween. Preferably, the monomers are C2-C8 compounds, more preferably C2-C6 compounds, and most preferably C2-C4 compounds such as ethylene, propylene and butylene.

Suitable hydrophilic unit(s) for polar modified polymers include, but are not limited to, maleic anhydride, acrylates, alkyl acrylates such as, for example, methyl acrylate, ethyl acrylate, propyl acrylate, and butyl acrylate, and polyvinylpyrrolidone (PVP).

In some embodiments, the oil-soluble high carbon polar modified polymer waxes can be used, including, e.g., C26-C28 alpha olefin maleic acid anhydride copolymer waxes commercially available from Clariant under the trade name LICOCARE or LICOCENE. Specific examples of such waxes include products marketed by Clariant under the LicoCare name having designations such as CM 401, which is a maleic anhydride modified wax having a Mw of 2025 and a crystallinity of 11%, C30-C38 olefin/isopropylmaleate/maleic anhydride copolymer sold by Baker Hughes under the name Performa® V 1608, and C24-C26 alpha olefin acrylate copolymer wax commercially available from Clariant under the trade name LICOCARE CA301 LP3346 based on a polar backbone with C24-26 side chains with alternating ester and carboxylic acid groups.

In some embodiments, C2-C3 polar modified waxes can be used, including, e.g., polypropylene and/or polyethylene-maleic anhydride modified waxes (“PEMA,” “PPMA.” “PEPPMA”) commercially available from Clariant under the trade name LICOCARE or LICOCENE, Specific examples of such waxes include products marketed by Clariant under the LicoCare name having designations such as PP207. Other suitable polar modified polymers include, but are not limited to A-C 573 A (ETHYLENE-MALEIC ANHYDRIDE COPOLYMER; Drop Point, Mettler: 106° C.) from Honeywell, A-C 596 A (PROPYLENE-MALEIC ANHYDRIDE COPOLYMER; Drop Point, Mettler: 143° C.) from Honeywell, A-C 597 (PROPYLENE-MALEIC ANHYDRIDE COPOLYMER; Drop Point, Mettler: 141° C.) from Honeywell, ZeMac® copolymers (from VERTELLUS) which are 1:1 copolymers of ethylene and maleic anhydride, polyisobutylene-maleic anhydride sold under the trade name ISOBAM (from Kuraray), polyisoprene-graft-maleic anhydride sold by Sigma Aldrich, poly(maleic anhydride-octadecene) sold by Chevron Philips Chemical Co., poly (ethylene-co-butyl acrylate-co-maleic anhydride) sold under the trade name of Lotader (e.g. 2210, 3210, 4210, and 3410 grades) by Arkema, copolymers in which the butyl acrylate is replaced by other alkyl acrylates (including methyl acrylate [grades 3430, 4404, and 4503] and ethyl acrylate [grades 6200, 8200, 3300, TX 8030, 7500, 5500, 4700, and 4720) also sold by Arkema under the Lotader name, and isobutylene maleic anhydride copolymer sold under the name ACO-5013 by ISP.

In some embodiments, the wax is an alcohol wax. “Alcohol wax” means a wax comprising at least one alcohol function, i.e. comprising at least one free hydroxyl (OH) group. When alcohol waxes are used, preferably, the at least one long-chain alcohol wax has an average carbon chain length of between about 20 and about 60 carbon atoms, most preferably between about 30 and about 50 carbon atoms. Suitable examples of long-chain alcohol waxes include but are not limited to alcohol waxes commercially available from Baker Hughes under the Performacol trade name such as, for example, Performacol 350 (INCI Name: C20-C40 alcohols), 425 (INCI Name: C20-C40 alcohols) and 550 (INCI Name: C30-050 alcohols). Most preferably, the long-chain alcohol wax has a melting temperature range from about 93° C. to about 105° C. Another example of a commercial product comprising a suitable alcohol wax is Performacol 550-L Alcohol from New Phase Technologies.

In some embodiments, the wax is an apolar wax. Apolar waxes are, in particular, hydrocarbon-based waxes formed solely from carbon and hydrogen atoms, and free of heteroatoms such as N, O and P. In particular, the term “apolar wax” means a wax that is formed exclusively from apolar wax and not from a mixture also comprising other types of waxes that are not apolar waxes.

Examples of apolar waxes include hydrocarbon-based waxes, for instance microcrystalline waxes, paraffin waxes, ozokerite and polyethylene waxes.

Polyethylene waxes that may be mentioned include Performalene 500-L Polyethylene and Performalene 400 Polyethylene sold by New Phase Technologies. An ozokerite that may be mentioned is Ozokerite Wax SP 1020 P. Examples of microcrystalline waxes include Multiwax W 445® sold by the company Sonneborn and Microwax HW® and Base Wax 30540® sold by the company Paramelt. Examples of microwaxes that may be used in the compositions according to the invention as apolar waxes include polyethylene microwaxes such as those sold under the names Micropoly 200®, 220®, 220L®, and 250S® by the company Micro Powders.

If present, waxes may be present in the composition in an amount ranging from about 0.1% to about 50%, preferably from about 1% to about 40%, and preferably from about 5% to about 30% by weight of the total weight of the composition, including all ranges and subranges therebetween.

In a preferred embodiment, the cosmetic composition uses a low level of waxes in the composition, where the total amount of wax is ≤6.5% by weight of the composition. In these embodiments, the wax is present in an amount between 3% and 6.5% by weight of the composition.

Hydrocarbons

The cosmetic composition may also comprise a hydrocarbon.

In some embodiments, the hydrocarbon is a hydrocarbon oil. In some embodiments, the hydrocarbon oil is a volatile hydrocarbon oil having from 8 to 16 carbon atoms and their mixtures and in particular branched C8 to C16 alkanes such as C8 to C16 isoalkanes (also known as isoparaffins), isododecane, isodecane, isohexadecane, and for example, the oils sold under the trade names of Isopar or Permethyl, the C8 to C16 branched esters such as isohexyl or isodecyl neopentanoate and their mixtures.

In some embodiments, the hydrocarbon may be a volatile hydrocarbon oil.

In some embodiments, the hydrocarbons comprise or consist of aliphatic hydrocarbons. Aliphatic hydrocarbons include, e.g., vaseline or petrolatum, naphthalenes, and the like; hydrogenated polyisobutene, isoeicosan, polydecenes, and decene/butene copolymer; and mixtures thereof. Examples of aliphatic hydrocarbons, can also include linear, branched, or cyclic C6-C16 lower alkanes, including hexane, undecane, dodecane, tridecane and isoparaffins such as isohexadecane and isodecane.

If present, hydrocarbons may be present in the composition in an amount ranging from about 0.1% to about 50%, preferably from about 1% to about 40%, and preferably from about 5% to about 20% by weight of the total weight of the composition, including all ranges and subranges therebetween.

Additional Polymer

The cosmetic composition may also include an additional cosmetically-acceptable polymer. This may be, e.g., thermoplastic elastomers such as Styrenic block copolymers (TPE-s), Thermoplastic olefins (TPE-o), Elastomeric alloys (TPE-v or TPV), Thermoplastic polyurethanes (TPU), Thermoplastic copolyester, Thermoplastic polyamides, Santoprene (ExxonMobil), Termoton (Termopol Polimer), Arnitel (DSM), Solprene (Dynasol), Engage (Dow Chemical), Hytrel (DuPont), Dryflex and Mediprene (ELASTO), Kraton (Kraton Polymers), Pibiflex, Forprene, Termoton-v, Sofprene (SBS) and Larprene (SEBS). In some embodiments, the polymer is a polymer based on starch, gum, and/or cellulose.

If present, fatty esters may be present in the composition in an amount ranging from about 0.1% to about 10%, preferably from about 0.5% to about 7%, and preferably from about 1% to about 5% by weight of the total weight of the composition, including all ranges and subranges therebetween.

Fatty Ester

The cosmetic composition may also include a fatty ester. The term “fatty ester” includes esters derived from a fatty acid and/or from a fatty alcohol. This may include a linear (unbranched) fatty ester, such as cetyl palmitate, as well as a branched fatty ester, such as ethylhexyl palmitate, isostearyl isostearate, etc.

In some embodiments, the fatty ester comprises an aromatic ester such as tridecyl trimellitate.

In some embodiments, the fatty ester comprises esters of a glycerol oligomer, especially diglycerol esters, in particular condensates of adipic acid and of glycerol, for which some of the hydroxyl groups of the glycerols have reacted with a mixture of fatty acids such as stearic acid, capric acid, stearic acid and isostearic acid, and 12-hydroxystearic acid, preferably such as bis-diglyceryl polyacyladipate-2 (INCI name) sold under the brand name Softisan 649 by the company Cremer Oleo,

If present, fatty esters may be present in the composition in an amount ranging from about 0.1% to about 50%, preferably from about 1% to about 40%, and preferably from about 5% to about 30% by weight of the total weight of the composition, including all ranges and subranges therebetween.

Non-Volatile Silicones

The cosmetic composition may also comprise a non-volatile silicone. Mention is made, as example of non-volatile silicone oils, of non-volatile polydialkylsiloxanes; polydimethylsiloxanes comprising pendant alkyl, alkoxy or phenyl groups or alkyl, alkoxy or phenyl groups at the end of the silicone chain, which groups have from 2 to 24 carbon atoms; phenylated silicones, such as those of the phenyl trimethicone type, those of the phenylpropyldimethylsiloxysilicate type or those of the trimethylpentaphenyltrisiloxane type; polysiloxanes modified by fatty acids, in particular C8-C20 fatty acids, fatty alcohols, in particular C8-C20 fatty alcohols, or polyoxyalkylenes (in particular polyoxyethylene and/or polyoxypropylene); aminated polysiloxanes; polysiloxanes comprising a hydroxyl group; and their mixtures.

If present, non-volatile silicones may be present in the composition in an amount ranging from about 0.1% to about 50%, preferably from about 1% to about 40%, and preferably from about 5% to about 20% by weight of the total weight of the composition, including all ranges and subranges therebetween.

In some embodiments, the cosmetic composition may be free, or substantially free, of certain materials. For example, in some embodiments, the cosmetic composition may be free or substantially free of UVA and/or UVB-absorbing components (UV filters), polyamides, acrylic polymers, hydrophilic synthetic amorphous silica particles, cellulose or ethylcellulose-based polymer particles, or a combination thereof. In a preferred embodiment, the disclosed combination of PHA and polysiloxane elastomer provides the functionality currently provided by, e.g., Nylon 12 or PMMA, and thus, in preferred embodiments, the composition is free or substantially free of Nylons (such as Nylon 12) and PMMA.

Once the composition is provided, the composition can be applied to lips in order to provide a matte color or mattifying effect to the lips.

### Example 1

Several lipstick compositions were created, as described in Table 1, below. A first comparative example (C1) used nylon 12, while the other formulations used various PHAs. Two comparative examples are shown (C1, C2) and three exemplary formulations (E1-E3). To produce each formulation, the pigments are grinded with a mixture of the non-volatile silicone oils, fillers, and fatty esters to great a pigment grind. The pigment grind and waxes are then placed in a kettle and heated up to 95° C. and mixed until homogenous. Once homogenous, the crosspolymers and the rest of the powders and antioxidants/fragrance are added and mixed until homogenous. Once completed, the hot formulations are poured into lipstick molds and cooled to room temperature before transferring to a chilled environment and then placed in lipstick packs.

TABLE 1

| Material | C1 | C2 | E1 | E2 | E3 |
|---|---|---|---|---|---|
| Antioxidants/ | 0.1-0.5% | 0.1-0.5% | 0.1-0.5% | 0.1-0.5% | 0.1-0.5% |
| Fragrance | | | | | |
| Hydrocarbons | 5-15% | 5-15% | 5-15% | 5-15% | 5-15% |
| Non-Volatile | 5-10% | 5-10% | 5-10% | 5-10% | 5-10% |
| Silicone Oils | | | | | |
| Fatty Esters | 20-30% | 20-30% | 20-30% | 20-30% | 20-30% |
| Waxes | 5-20% | 5-20% | 5-20% | 5-20% | 5-20% |
| PDMS | — | 21-35% | | | |
| Polysiloxane | 1-5% | — | 1-5% | 1-5% | 1-5% |
| Elastomer (Vinyl | | | | | |
| Dimethicone/ | | | | | |
| Methicone | | | | | |
| Silseqsquioxane | | | | | |
| Crosspolymer) | | | | | |
| Additional | 20-30% | — | 20-30% | 20-30% | 20-30% |
| polysiloxane | | | | | |
| elastomer | | | | | |
| (dimethicone and | | | | | |
| dimethicone | | | | | |
| crosspolymer) | | | | | |
| Nylon 12 | 1-5% | — | — | — | — |
| Amorphous Silica | — | 1-5% | — | — | — |
| Hydroxybutyric | — | — | 1-5% | — | — |
| acid/hydroxypent | | | | | |
| anoic acid copolymer | | | | | |
| (10 μm particles) | | | | | |
| 3-Hydroxybutyrate/ | — | — | — | 1-5% | — |
| 3-Hydroxyhexanoate | | | | | |
| Copolymer (20 μm | | | | | |
| particles) | | | | | |
| PHB (10 μm | — | — | — | — | 1-5% |
| particles) | | | | | |
| Pigments/Fillers | 10-20% | 10-20% | 10-20% | 10-20% | 10-20% |

Evaluations

The various lip products were evaluated for, finish (shiny or matte), glide smoothness, dispersion, and feel.

TABLE 2

| Test | C1 | C2 | E1 | E2 | E3 |
|---|---|---|---|---|---|
| Finish | Matte | Shiny | Matte | Matte | Matte |
| (Shiny/Matte) | | | | | |
| Glide | Good | Good | Good | Good | Good |
| Smoothness | | | | | |
| Dispersion | Ok | Poor | Good | Good | Good |
| | | (Undispersed | | | |
| | | Globs) | | | |

As seen in the table above, the exemplary compositions provide a matte finish, good glide smoothness, and good dispersion, as compared to the comparative formulations. Of particular note, replacing the PHAs with other particulates (e.g., amorphous silica, see C2) resulted in small, undispersed globs appearing after application.

Further, it is noted that when replacing the PHAs with hydrophilic synthetic amorphous silica particles and using dimethicone (PDMS) as opposed to the two listed crosspolymers, resulted in shiny (as opposed to matte) finish, and poor dispersion of the pigments and fillers.

### Example 2

Formulations were also created to compare pigment grinds and PHAs alone versus the pigment grinds and PHAs in combination with the polysiloxane elastomer. For example, samples were created using various pigments and PHB alone, as well as samples using those pigments and PHB combined with Vinyl Dimethicone/Methicone Silsesquioxane Crosspolymer (KSP-100, Shin-Etsu). Each sample was then evaluated via a drawdown test where a small sample is placed at the top end of a Hegman gauge and then drawn down with a flat edge along the grooves.

The drawdown of the pigment grind with PHB alone began streaking at approximately 25 μm, and heavy streaking was seen at 20 μm and below. When the KSP-100 was added, streaking did not begin until approximately 20 μm, with only 3-4 streaks in total.

Thus, it is clear that the silicone elastomer, in combination with the PHAs, improves the dispersion of particulates, such as pigments and fillers, in formulations. In particular, the disclosed combination improves dispersion in formulations that are not inherently free-flowing, such as in pasty, waxy, or solid cosmetic compositions.

### Example 3

Various example formulations can be created at different wax levels, see Table 3, below.

TABLE 3

| Material | E4 | E5 | E6 | E7 | E8 |
|---|---|---|---|---|---|
| Antioxidants/ | 0.1-0.5% | 0.1-0.5% | 0.1-0.5% | 0.1-0.5% | 0.1-0.5% |
| Fragrance | | | | | |
| Volatile | 5-15% | 5-15% | 5-15% | 5-25% | 5-50% |
| Hydrocarbon Oils | | | | | |
| Non-Volatile | 5-10% | 5-10% | 5-10% | 5-10% | — |
| Silicone Oils | | | | | |
| Fatty Esters | 20-30% | 20-30% | 20-30% | 20-30% | — |
| Waxes | 0.1-0.5% | 0.5-1% | 3-6.5% | 3-6.5% | 3-30% |
| Additional Polymer | 0.1-10% | 0.1-10% | 0.1-10% | 0.1-10% | — |
| Polysiloxane | 0.1-8% | 0.1-8% | 0.1-8% | 0.1-8% | 0.1-8% |
| Elastomer (Vinyl | | | | | |
| Dimethicone/ | | | | | |
| Methicone | | | | | |
| Silseqsquioxane | | | | | |
| Crosspolymer) | | | | | |
| Additional | 1-30% | 1-30% | 1-30% | — | — |
| polysiloxane | | | | | |
| elastomer | | | | | |
| (dimethicone and | | | | | |
| dimethicone | | | | | |
| crosspolymer) | | | | | |
| PHB (10 μm | 0.1-30% | 0.1-30% | 0.1-30% | 0.1-30% | 0.1-30% |
| particles) | | | | | |
| Pigments/Fillers | 10-20% | 10-20% | 10-20% | 10-20% | 10-20% |

Those skilled in the art will recognize or be able to ascertain using no more than routine experimentation many equivalents to the specific embodiments of the invention described herein. Such equivalents are intended to be encompassed by the following claims

What is claimed is:

1. An anhydrous cosmetic composition, comprising:
a polyhydroxyalkanoate;
a polysiloxane elastomer; and
a filler, a pigment, or both.

2. The anhydrous cosmetic composition according to claim 1, wherein the polysiloxane elastomer consists of an elastomeric crosslinked organopolysiloxane powder coated with silicone resin.

3. The anhydrous cosmetic composition according to claim 2, wherein the silicone resin is a silsesquioxane resin.

4. The anhydrous cosmetic composition according to claim 2, wherein the polyhydroxyalkanoate comprises (i) a polyhydroxybutyrate, (ii) poly [(R)-3-hydroxypentanoate] (PHHP), (iii) poly [(R)-3-hydroxyhexanoate] (PHHx), (iv) a crosspolymer comprising monomers from hydroxybutyric acid, hydroxyhexanoic acid, and/or hydroxyheptanoic acid, or (v) a combination thereof.

5. The anhydrous cosmetic composition according to claim 1, wherein the polyhydroxyalkanoate is present in an amount of between 1% and 5% by weight of the composition.

6. The anhydrous cosmetic composition according to claim 1, wherein the polyhydroxyalkanoate has a particle diameter (d50) of 8 μm to 12 μm.

7. The anhydrous cosmetic composition according to claim 6, wherein the polyhydroxyalkanoate has a particle diameter (d50) is 10 μm.

8. The anhydrous cosmetic composition according to claim 1, wherein polysiloxane elastomer is present in an amount of between 1% and 5% by weight of the composition.

9. The anhydrous cosmetic composition according to claim 1, wherein the cosmetic composition is a solid lipstick.

10. The anhydrous cosmetic composition according to claim 1, wherein the cosmetic composition is substantially free of UV filters.

11. The anhydrous cosmetic composition according to claim 1, wherein the cosmetic composition is substantially free of hydrophilic synthetic amorphous silica particles.

12. The anhydrous cosmetic composition according to claim 1, wherein the cosmetic composition is substantially free of polyamides.

13. The anhydrous cosmetic composition according to claim 1, wherein the cosmetic composition is substantially free of acrylic polymers.

14. The anhydrous cosmetic composition according to claim 1, wherein the cosmetic composition is substantially free of cellulose or ethylcellulose-based polymer particles.

15. The anhydrous cosmetic composition according to claim 1, further comprising a silicone crosspolymer in amount of between 5% and 30% by weight of the composition.

16. The anhydrous cosmetic composition according to claim 1, further comprising a wax and a hydrocarbon oil.

17. The anhydrous cosmetic composition according to claim 1, further comprising a filler, an antioxidant, a fragrance, a colorant, a wax, a volatile hydrocarbon oil, an additional polymer, a fatty ester, a non-volatile silicone, or a combination thereof.

18. The anhydrous cosmetic composition according to claim 1, further comprising a wax in an amount of between 3% and 6.5% by weight of the composition.

19. The anhydrous cosmetic composition according to claim 1, wherein the anhydrous cosmetic composition consists essentially of:
the polyhydroxyalkanoate;
the polysiloxane elastomer;
the pigment; and
optionally a filler, an antioxidant, a fragrance, a colorant, a wax, a hydrocarbon, an additional polymer, a fatty ester, a non-volatile silicone, or a combination thereof.

20. A method for providing matte color or effect to lips, comprising:
providing an anhydrous cosmetic composition comprising a polyhydroxyalkanoate; a polysiloxane elastomer; and a pigment, filler, or both; and
applying the anyhydrous cosmetic composition to lips.
